(19) United States
(12) Patent Application Publication
(10) Pub. No.: US 2024/0374241 A1
(43) Pub. Date: Nov. 14, 2024

(54) DEVICE AND METHOD FOR THE DIAGNOSIS OF A PNEUMONIA BY FREQUENCY ANALYSIS OF ULTRASOUND SIGNALS

(71) Applicant: Imedicals S.r.l., Lecce (IT)

(72) Inventor: Sergio Casciaro, Lecce (IT)

(73) Assignee: Imedicals S.r.l., Lecce (IT)

(21) Appl. No.: 18/289,447

(22) PCT Filed: May 4, 2021
(86) PCT No.: PCT/IB2021/053702

§ 371 Date: Nov. 3, 2023

(51) Int. Cl. A61B 8/08; A61B 8/00; G06T 7/00; G06T 7/11

(52) U.S. Cl. CPC A61B 8/5223; A61B 8/4483; A61B 8/5207; A61B 8/5269; G06T 7/0014; G06T 7/11; G06T 2207/10132; G06T 2207/20021; G06T 2207/20084; G06T 2207/30061; G06T 2207/30204

(57) ABSTRACT

A method for calculating a diagnostic parameter indicating the stage of a pneumonia, comprising the steps of: (100) acquiring an ultrasound image of the lung, with the pleural line and a portion below it; (200) individuating the region under the pleural line; (300) segmenting said region individuate a set of ultrasound makers (C1, . . . , Cn); (310) individuating at least a region of interest (ROI) as a function of the type, quantity and configuration of the ultrasound markers individuated at step (300); (450) extracting frequency spectra from raw ultrasonic signal corresponding to segments of the ultrasound image contained in each ROI individuated at step (310): (470) comparing each one of said spectra with relative reference spectra calculated for healthy patients and for patients suffering from pneumonia at various stages and calculate a plurality of correlation parameters; (500) calculating a diagnostic parameter as a function of said calculated at point (470).

### BACKGROUND OF THE INVENTION

### 1. Field of the Invention

The present invention relates to an ultrasound images and relative “raw” ultrasonic signals analysis, configured to allow a numeric diagnostic parameter automatized calculation, indicating the possible presence and the progression stages of pneumonia, while distinguishing also a COVID-19 type pneumonia, i.e. caused by SARS-CoV-2 virus, from other pneumonia types, which can be individuated specifically and by means of the same method as well.

### 2. Brief Description of the Prior Art

Thanks to their high sensibility, chest X-ray and computed tomography (CT) are currently the imaging techniques of choice used to diagnose and monitor patients with general pneumonia, as for example also the one caused by COVIV-19 (Coronavirus Disease caused by SARS-CoV-2).

However, both chest X-ray and CT have some important limitations due to limited accessibility, high ionizing radiation doses and high costs, which do not allow their usage for screening purposes. Moreover, it could be difficult for patients suffering from hypoxemia and/or heart failure to have a CT, and it is also difficult to transfer patients from intensive care unit to the CT system and, anyway due to the connected infection risks important transfer limitations remain.

Lung ultrasound has shown a promising capacity of COVID-19 patient diagnosis and monitoring, similar to chest CT and superior to standard chest radiography used to evaluate pneumonia and/or adult respiratory distress syndrome. Lung ultrasound can overcome the main CT limitations, since it can be brought to patient's home or bed, it is accurate, radiations-free, cheaper, and so it could become the imaging method of choice for patients, whose triage has to be carried out at home or in critical conditions in intensive care units, or however for old patients and for specific categories, such as pregnant women and children.

It is known (Peng et al., “Findings of lung ultrasonography of novel coronavirus pneumonia during the 2019-2020 epidemic”-Intensive Care Med 2020) that COVID-19 pneumonia, on the ultrasound images obtained by scanning various points of the rib cage, with specific diffusion “patterns” produces:
a pleural line thickening, with irregularities,
pleural effusions, a plurality of vertical lines, called B-lines (or also “lung comets”), which can be focal, multi focal or confluent; consolidation areas (also called “consolidative areas” or “consolidations”) in a plurality of different configurations (patterns) including small multi-focal consolidations areas and not trans-lobular wider (also called “intra-lobular”), trans-lobular (also called “inter-lobular”) consolidation areas with possible dynamic air bronchograms.

A lung consolidation is observed when the lung ventilation degree is reduced, and air is substituted by exudate/inflammatory cells in the alveoli. Thus, the ultrasound beam is able to penetrate the parenchyma highlighting a “consolidation” with ultrasound pattern structure similar to the liver one (pulmonary hepatization). In the B-mode ultrasound, the pulmonary parenchymal consolidation is associated to the following phenomena:
pleural line interruption/interruptions;
presence of one or more homogeneous hypoechoic areas (similar to liver tissue) below the pleural line interruptions; punctiform and/or linear hyperechoic areas (called “air bronchograms”) on the deepest edge of the hypoechoic area and/or inside the same; elongated white areas with “cascade” effect, which go from hyperechoic areas to the bottom of the B-Mode image.
The above-described effects are amplified as a function of the disease severity, as it is schematized for simplicity and as a way of example in the following table which describes in detail the aspect of the different stages of pulmonary consolidation on the ultrasound images:

| | | Hypoechoic | Hyperechoic | |
|---|---|---|---|---|
| stage | Pleural line | area | areas | Cascade effect |
| 1 | One or two | Non- | Little areas | Visible with |
| | interruptions of | essential | present, | moderate |
| | small dimensions, | or little | very close | intensity, |
| | visible in the | extended | to pleural | starting very |
| | B-mode field of | area | line | close to pleural |
| | view | | | line |
| 2 | At least two | One or more | One or more areas | Increase of |
| | visible | areas with | clearly not | intensity and of |
| | interruptions of | greater | adjacent to | extension of the |
| | pleural line. | extension | pleural line | effect |
| | Dimensional | | | |
| | increase of the | | | |
| | interruptions | | | |
| 3 | Unification and | Very | Many | High intensity. |
| | increase of | extended | punctiform, | Start very far |
| | interruptions; | area | spot and/or | from pleural line |
| | increase of | | linear | |
| | pleural line | | areas, both | |
| | interrupted | | inside the | |
| | portion | | hypoechoic | |
| | | | area and on | |
| | | | the deepest | |
| | | | edge | |

Moreover, during the recovering stage from COVID-19 pneumonia, on the ultrasound images it is also possible to observe:

“A-lines” occurrence (substantially horizontal lines, or anyway perpendicular to the propagation direction of the ultrasonic beam emitted by the probe and substantially parallel to the pleural line).

In FIG. 1, there are shown some lung ultrasound images of a patient with confirmed COVID-19 pneumonia.

In the two upper images, typical vertical wide artifacts can be observed, which start from pleural line or small sub-pleural consolidations. The vertical artifacts origin is not punctual. In the lower images, it is observed how the pleural line is interrupted by more visible consolidations. It is observed that from these consolidations vertical wide artifacts start, overlapping with a white lung area. Further ultrasound images relative to a patient with confirmed Coronavirus pneumonia are shown in FIG. 2.

In a different field of application of the signal analysis techniques (WO2012/156937) it is known the use of the analysis of frequency spectra relating to bone tissues to evaluate the presence of osteoporosis. Anyway, the ultrasound principles applied to bone tissues are not directly applicable to the lung ultrasound, and so also the method described in WO2012/156937 is not directly applicable in the case of lung ultrasound. In fact, it is known that in case of lung ultrasound, on the ultrasound image a series of artifacts are provided which make the segmentation in the time domain described in such document not efficient to individuate significant Regions of Interest. Moreover, in case of bone tissue the RF signals of the Region of Interest derive directly from ultrasonic signal backscatter generated by a given region of bone tissue, and so the study of the characteristics of these signals can lead to determining specific characteristics of the tissue portion visualized in the corresponding image portion. In case of lung ultrasound, instead, the most part of the markers visible on the ultrasound images correspond to “artifacts”.

For example, A-lines are generated by multiple reflections of the ultrasonic signal bouncing between pleura and probe surface (the A-lines located at the bottom of the image do not correspond to anatomical structures located in the lung depth, but always to pleura reflections); B-lines, in turn, are due to the presence of water just under the pleura (in the interstice) and their appearance on the image is generated actually by the ultrasonic signal continuous bouncing in the water at the air interface (also in this case, the presence of a B-line reaching the bottom of the image does not represent an anatomical structure located in the corresponding region of the image, but it is generated by a signal many times reflected in the water volume located close to the pleura).

Therefore, in case of lungs, the RF signal analysis is needed to characterize accurately and quantitively the characteristics of markers visible on the image (by an expert eye) and in particular, in comparison to the case of bone tissue (and other soft tissues different from lung), the correspondence between the RF signal characteristics and the characteristics of the anatomical region located at the portion of the image obtained by the same RF signal goes completely lost.

### Technical Problem

According to the state of the art, the usage of lung ultrasound has various limitations, as it is explained for example in Bouhemad et al., “Clinical review: Bedside lung ultrasound in critical care practice”, Crit Care. 2007; 11 (1): 205.

In primis, an irregular B-line or a consolidation pattern can be observed in any pneumonia or interstitial lung disease, even not associated to COVID-19, and it is nearly impossible, even for a skilled healthcare operator, to distinguish between the various disease types only on the basis of the subjective image analysis.

The lung ultrasound requires also an intensive training on simple applications of at least six weeks of the healthcare operator in order to allow him to acquire the needed knowledge and skills; moreover, currently, there are no quantitative indicators available deriving from the images, and so the diagnosis remains of qualitative type and its reliability depends strongly on the operator expertise.

In case of lungs, unlike others, the RF signal analysis does not give spatial information on the localization of the markers characteristic of the disease or physiologic condition quantified by means of the same RF signal analysis.

### SUMMARY OF THE INVENTION

Therefore, object of the present invention is a method of ultrasound images and relative unfiltered ultrasonic signals (so called “raw” or “radiofrequency” ultrasonic signals) analysis, which allows to obtain a quantitative evaluation of lung tissues condition. The present invention provides also an ultrasound device comprising computing means on which computer programs, configured to carry out such method, are loaded.

Yet, the invention provides a method of lung ultrasound images and relative unfiltered ultrasonic signals analysis, configured to calculate at least one quantitative diagnostic parameter, indicating the possible presence of a lung disease, whether it is caused by SARS-CoV-2 virus or by any other cause, and its clinical stage.

According to another aim, the present invention provides a method of lung ultrasound images and relative ultrasonic signals analysis which has all the just described advantages and whose results are highly repeatable and independent of the operator expertise.

One of the advantages of the method according to the present invention is that the same can be implemented by means of computer programs loaded on computing means associated to an ultrasound device, and so patients can be examined both at home and at the hospital and on ambulances as well as in any other structure provided for emergency, thanks to the portability of the diagnostic device, which can be always used also directly at the bed of the patient.

Another advantage of the present invention is that the method according to the invention can be implemented also on remote computing means (i.e. not integrated in the ultrasound device) to which the ultrasound images and/or the RF ultrasonic signals are provided in input. In this manner, the method can be implemented also by means of ultrasound devices yet available in the healthcare facilities, only configuring the same so that the images and/or relating ultrasonic signals are exported.

Another advantage is that the method according to the invention does not require skilled ultrasound operators for its own implementation, since the method provides quantitative diagnostic indicators calculated in a fully automatic manner and independent of the operator.

Another advantage is that the ultrasound acquisition for implementing the method follows a very easy protocol, during whose execution the operator is guided by the software and in which the acquisitions not corresponding to the protocol criteria are automatically rejected, and the operator is asked to repeat them.

In particular, the diagnostic indicators calculated with the method according to the invention allow to characterize the pneumonia, by defining if it is caused by COVID-19 or by any other type of virus or other causes (for example, bacteria, parasites, fungi, chronic obstructive pulmonary disease (COPD), etc.).

Another advantage is that the quantitative diagnostic indicators calculated by means of the method according to the invention allow an objective disease severity staging and the early identification of COVID-19 possible presence before the onset of pulmonary fibrosis in asymptomatic patients.

Finally, one of the main advantages of the method according to the invention is to allow to monitor the disease progression in a determined patient. In fact, the quantitative diagnostic indicators calculated by means of the method according to the invention comprise not only a descriptive indication of the disease “staging” (mild, moderate, severe, etc.), but also a specific numeric value (Pneumonia Score). For example, assuming that Pneumonia Score is indicated as numeric parameter in a scale from 1 to 100, having the disease in the “moderate” stage with Pneumonia Score=55 is different from having it in the same “moderate” stage but with Pneumonia Score=50. If the two values were obtained on the same patient, depending on which one was obtained at first, it will be possible to understand if the disease is in progression or in remission and also, as a function of the time interval between the two acquisitions, at what speed.

As an alternative, Pneumonia score can be indicated in a scale from 0 to 4, by classifying each lung tissue portion (or the patient as a whole) as healthy, or with the disease in the initial, intermediate, advanced or critical stage.

The possibility to repeat the acquisition every day (and basically also more times a day), combined with the availability of said quantitative diagnostic indicator, allows to carry out a “short term” monitoring and a rapid individuation of the disease progression trend, which is unthinkable for any other technology.

Moreover, a short term monitoring of the just described type is extremely useful to individuate the correct patient management, also considering the limited beds in intensive care units, and to evaluate as well the actual effectiveness of a drug, also in comparison with patients treated with different approaches (due to the usage of a different drug or because administration doses/times of the same drug vary), and this would have a fundamental importance in the clinical studies aiming at the introduction of new drugs.

### BRIEF DESCRIPTION OF THE DRAWINGS

FIGS. 1 and 2 show lung ultrasound images, with convex probe intercostal (transversal) acquisition relating to patients with COVID-19 pneumonia, and indication of consolidations and B-lines position; FIG. 3 shows a lung ultrasound image on which the pleural line identification is indicated; FIGS. 4 and 5 show two ultrasound images with indication of A-lines and B-lines, respectively; FIGS. 6 to 9 show flowcharts illustrating the steps needed to carry out the method according to the present invention; FIG. 10 shows an overall flowchart of the method for calculating a diagnostic parameter according to the invention; FIGS. 11, 12 and 13 show three examples of ultrasound images of the COVID-19 disease progression and relating in particular to three progressive advancement stages of lung consolidation.

### DETAILED DESCRIPTION OF THE PREFERRED EMBODIMENTS

### Term and Definitions

It is to be said at first that in the context of the present Patent application, for “raw ultrasonic signal” or “radiofrequency ultrasonic signal” is intended an ultrasonic signal emitted by the probe and reflected by the human body towards the same probe, before the same is processed in order to obtain the ultrasound image; yet, it is to be specified that, where not stated otherwise, for “ultrasound image” it is intended an ultrasound image of B-mode type, obtained along the propagation plane of the ultrasonic beam emitted by the probe.

Yet, it is to be specified that for “raw ultrasonic signal corresponding to a determined Region of Interest (ROI)” it is intended the portion of the raw ultrasonic signal which, suitably treated, has given origin to a corresponding segment of the Region of Interest identified on the B-mode ultrasound image.

As it is known, in fact, the correlation between the position of each pixel in the ultrasound image and the ultrasonic signal which generated it, is a function of the time interval occurring between the ultrasonic impulse emission and the relative echo reception (reflected signal), since the signal reflected by tissues positioned at greater depths needs more time to reach the probe after being reflected.

Therefore, regardless of the nature of possible subsequent processing, the segmentation of the “raw ultrasonic signal corresponding to a determined ROI” occurs in the time domain, so that the portion of the raw ultrasonic signal that, in the ultrasound image, has given origin to a determined segment of the same image is isolated. Yet, according to what commonly known at the state of the art, the used ultrasonic probe comprises an array of CMUT or piezoelectric type transducers arranged side by side, configured to emit a plurality of ultrasonic signals, so that a “line of sight” of the ultrasound image (directed up-downwards) corresponds to each signal, and the group of lines of sight, arranged side by side, allows to reassemble the ultrasound image. It is to be specified that the processing carried out on the radiofrequency ultrasonic signals is described in the following, for shortness and description clarity's sake, with reference to a single ultrasonic signal. Obviously, also where not specifically stated, it is clear that the processing as a whole can be conveniently applied to a plurality of raw ultrasonic signals, each one received by one of the CMUT or piezoelectric transducers, included in the ultrasound probe.

It is to be precise that for “point” of the raw signal (radiofrequency ultrasonic signal), it is intended the value assumed by the raw signal in a single sampling: in a sampling at 40 MHz, as a way of example, 40,000 points are obtained for each millisecond of acquired signal.

Moreover, it is to be specified that the whole processing described in the following is carried out by means of an ultrasound system provided with at least one ultrasound probe-which can be both of convex type or linear type or also of trans esophageal or matrix phased array type—and with suitable guiding means of said probe, with computing means for processing the signal, configured to generate the signal to be sent by means of said probe, and to analyze the signal received by said probe in order to obtain an ultrasound image, with user interaction means comprising a graphical interface and control means, as for example keyboards and/or pointing means. These are, also as a whole, the devices known at the state of the art and commonly used in the ultrasound technique. Anyway, it is to be precised that, in order to implement the diagnostic methods requiring a radiofrequency ultrasonic signal usage, the ultrasound device according to the invention is configured not only to process the raw ultrasonic signal (radiofrequency ultrasonic signal) to obtain an ultrasound image but also to store the raw ultrasonic signal in order to carry out following processing of the same.

Yet, it is useful to precise that in the present description, the definition “Ultrasound Dataset” refers to all the radiofrequency ultrasonic signals relating to a plurality of sequentially acquired frames of a specific patient.

From the ultrasound signals relating to each frame, according to the processing commonly known at the state of the art, the corresponding ultrasound image can be reassembled. Anyway, the raw ultrasonic signal contains further information which goes normally lost during the processing needed to obtain the ultrasound image, and so not present in the image, but which can be conveniently used to improve the efficacy of the diagnostic method according to the present invention, as explained in detail in the following. The definition “spectrum associated to a segment of ultrasound image” refers to the frequency spectrum obtained by the transformation of the raw ultrasonic signal corresponding to a respective segment of the ultrasound image.

### Method for Computing the Diagnostic Parameter

In the following, with reference to some preferred embodiments, the diagnostic method according to the invention is described, which can be implemented by means of a device of the just described type.

The method according to the invention comprises the steps of:

(100) acquiring at least one ultrasound image of the lung of the patient, in which at least the pleural line and a portion of the lung below it are visible.

Said at least one image is preferably an image acquired according to a technique commonly known as B-Mode Imaging, by means of an ultrasound system provided with an ultrasound probe comprising an array of CMUT or piezoelectric transducers, each transducer being configured to emit a ultrasonic impulse directed towards the tissues object of classification and to receive the raw ultrasonic signal reflected by the tissues of the patient in response to said ultrasonic impulse; preferably, moreover, both the radiofrequency raw ultrasonic signal received by said transducers and said at least one ultrasound image are saved.

Preferably, moreover, the method provides to consider each acquired image as acceptable for the following processing as a function of an automatized control, implemented by means of suitable programs loaded on computing means associated to said ultrasound device and configured to carry out the following operations:

(i) individuating the pleural line (whose ultrasound track is present both in healthy patients and in those with lung diseases) in the first 4 centimeters of the image depth and carrying on with the following controls only in case of effective individuation of the pleural line, thus rejecting the image otherwise.

Even if other implementations are possible, it is to be specified that the pleural line can be individuated by:
(a) applying a “Sobel” type filter to improve the visibility of the horizontal structures;
(b) applying an “Otsu” type threshold, in order to highlight the clearer structures;
(c) carrying out two cycles of: (i) horizontal smoothing of the image, in order to highlight further the horizontal structures as the pleura;
(ii) linear erosion, in order to remove the corpuscular and not-elongated appearing structures;
(d) identifying, starting from the bottom, the residual horizontal (continuous or patchy) structure with higher intensity;
(e) interpolating the recognized structure with a second-degree polynomial.

After individuating the pleural line, the next steps are:
(ii) verifying that the pleural line length is greater than a percentage threshold of the B-mode image width (preferably between 60% and 70%), thus rejecting the image otherwise;
(iii) verifying that the pleural line thickness is lower than a predetermined threshold (for example 5 mm), thus rejecting the image otherwise;
(iv) verifying that the pleural line average intensity is greater than a determined threshold
(as a way of example, a grey average intensity >200, for 8-bit images), thus rejecting the image otherwise;
(v) in case the pleural line is detected and the previous controls are satisfied, generating normalized histograms (i.e. with total area=1) of the shades of grey relating to the tissues above and below the pleural line, and carrying out the following controls (example numeric thresholds are indicated, which can be applied in case of images acquired with “transversal” probe positioning, i.e. the probe is parallel to ribs and positioned in the intercostal space; in case of “longitudinal” positioning, i.e. with probe perpendicular to the ribs, it is possible to carry out controls with the same approach but with different numeric threshold values): a. verifying that the portion of the histogram relating to the tissues above the pleura, representing the darkest shades of grey (for example, grey with intensity 0 to 25, on an 8-bit scale) is greater than a determined fraction of the whole area (for example 0.10), thus rejecting the image otherwise; b. verifying that the portion of the histogram relating to the tissues above the pleura, representing the lightest shades of grey (for example, grey with intensity 230 to 255, on an 8-bit scale), is greater than a determined fraction of the whole area (for example 0.10), thus rejecting the image otherwise; c. verifying that the portion of the histogram relating to the tissues below the pleura, representing the intermediate shades of grey (for example, grey with intensity 50 to 200, on an 8-bit scale), is greater than a determined fraction of the whole area (for example 0.9), thus rejecting the image otherwise.

Thus, the following processing described in the following is carried out only on images not rejected after said controls.

Preferably, the device according to the invention comprises also a graphical interface and is configured to communicate to the operator, by means of said graphical interface, if the acquired image has been validated or not (i.e. if the acquisition satisfies the protocol requirements and if the relative ultrasound dataset is then suitable to be analyzed to provide a diagnostic result or not). Therefore, the method comprises the steps of:

(200) individuating, in said at least one image acquired at point (100) a region containing a significant lung tissue portion.

Preferably, said region comprises the whole area under the pleura, also because the pleura is a visible structure both in healthy and ill patients.

It is to be specified that said significant portion comprises a portion of lung parenchyma, the region of lungs around the bronchus, formed by the whole pulmonary lobules.

(300) Segmenting the region under the pleural line in order to individuate a set of ultrasound markers (Ci, . . . , Cn) therein.

Conveniently, the segmentation can be carried out by means of automatic image segmentation routines. The logic used to carry out this segmentation is explained in the following, for each ultrasound marker. The computing implementation of these logics, once the same are stated, can be realized with various tools, among the ones known at the state of the art. The ultrasound markers, object of the segmentation, are described in the following.

Ci: pleural line (whose identification on a lung ultrasound image is shown in FIG. 3). In order to obtain an automatic segmentation algorithm, the pleural line can be conveniently identified as the horizontal interface with greater contrast and/or absolute brightness present on the ultrasound image. A possible automatic method of pleural line individuation was explained previously. C2: A-lines (whose identification on a lung ultrasound image is shown in FIG. 4). Where present, A-lines can be identified by means of an automatic segmentation algorithm which, once the pleural line is detected, analyzes the image from the pleural line downwards by using horizontal gradient filters and contrast masks.

C3: B-lines (whose identification on a lung ultrasound image is shown in FIG. 5). Where present, B-lines can be identified by means of an automatic segmentation algorithm which, once the pleural line is detected, carries out the analysis downwards and by using vertical gradient filters and contrast masks.

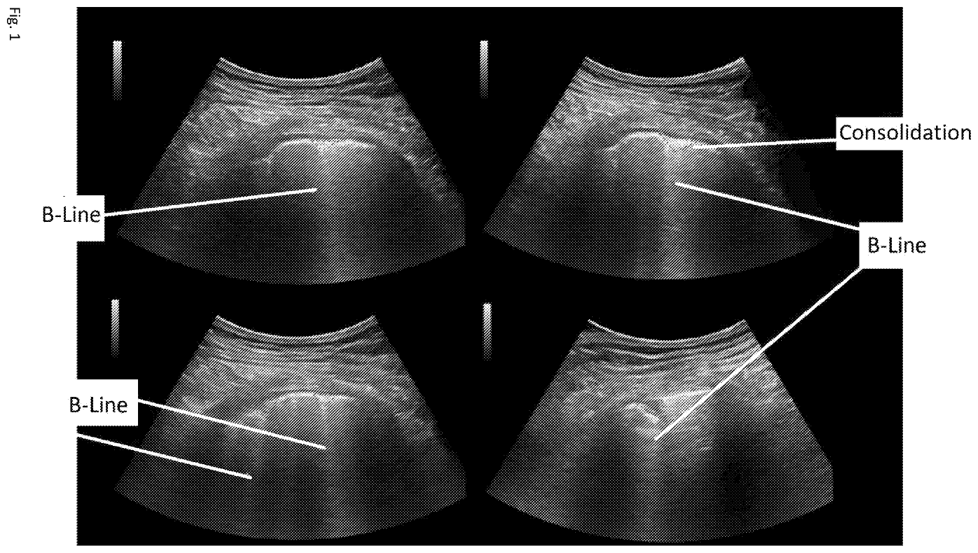

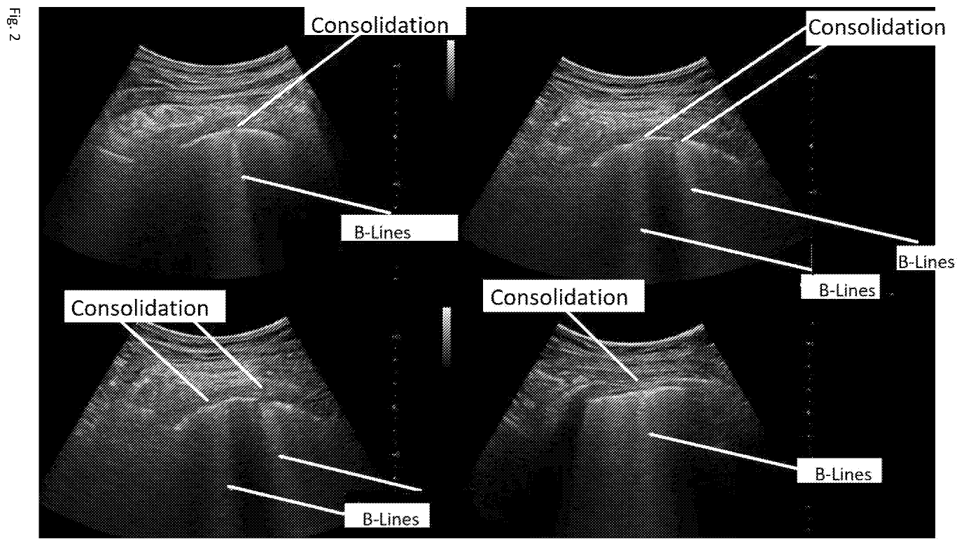

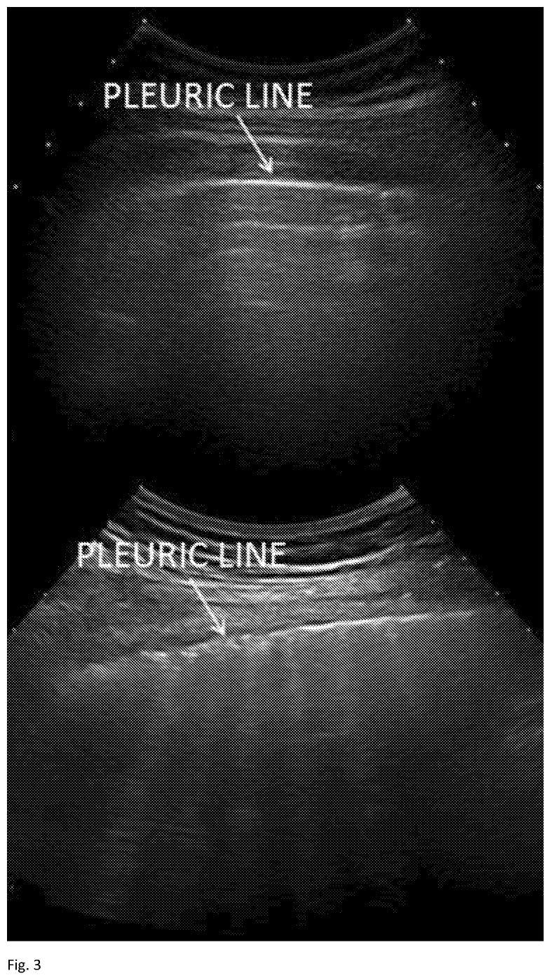

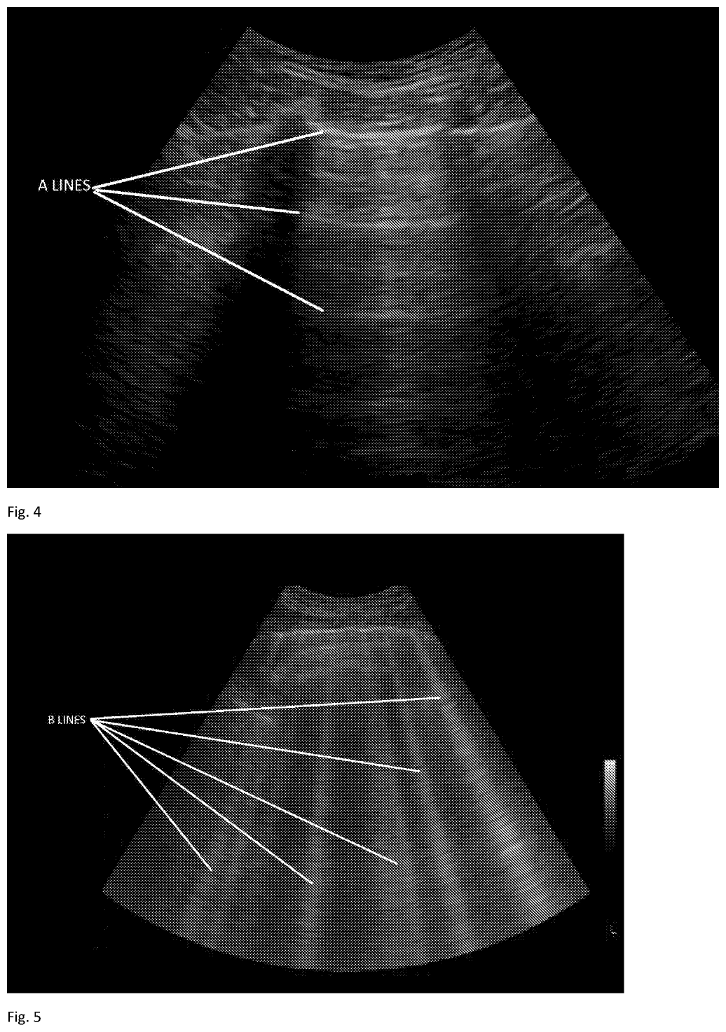

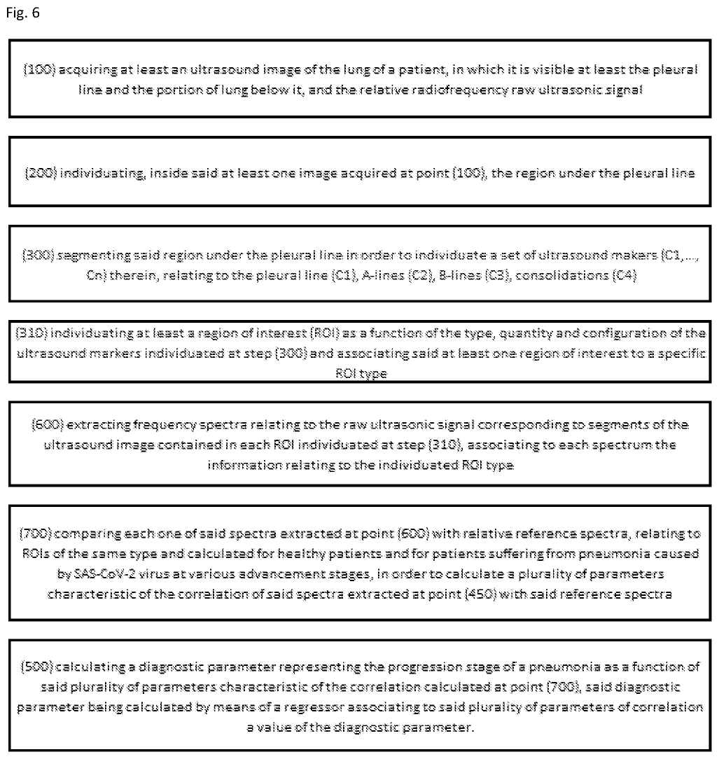

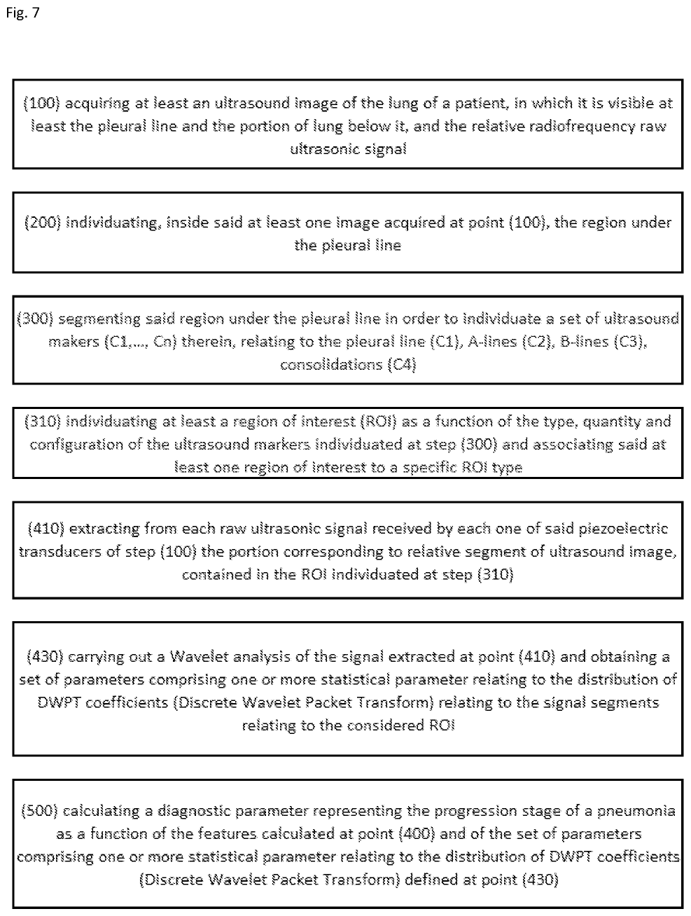

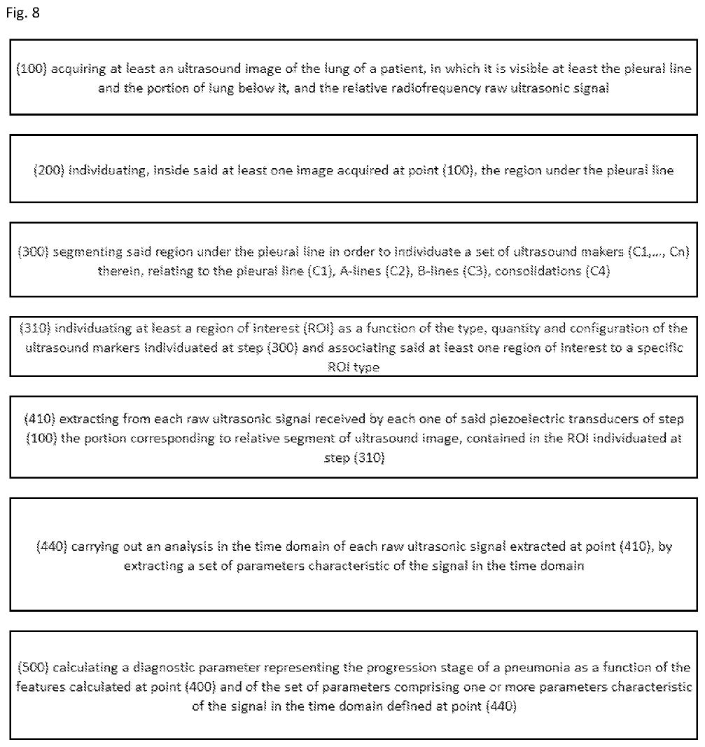

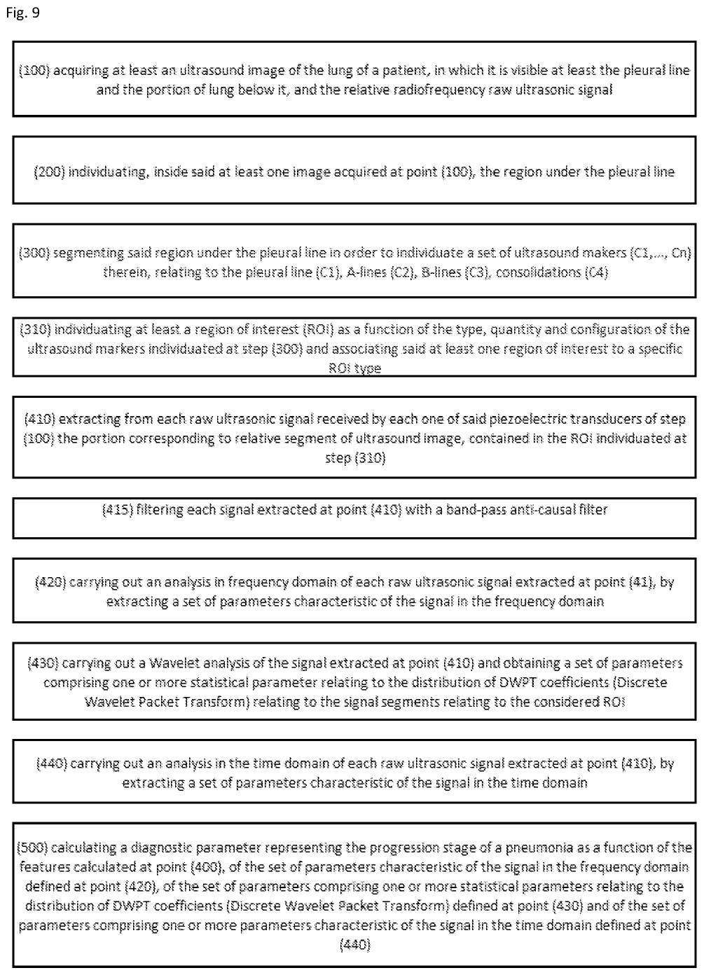

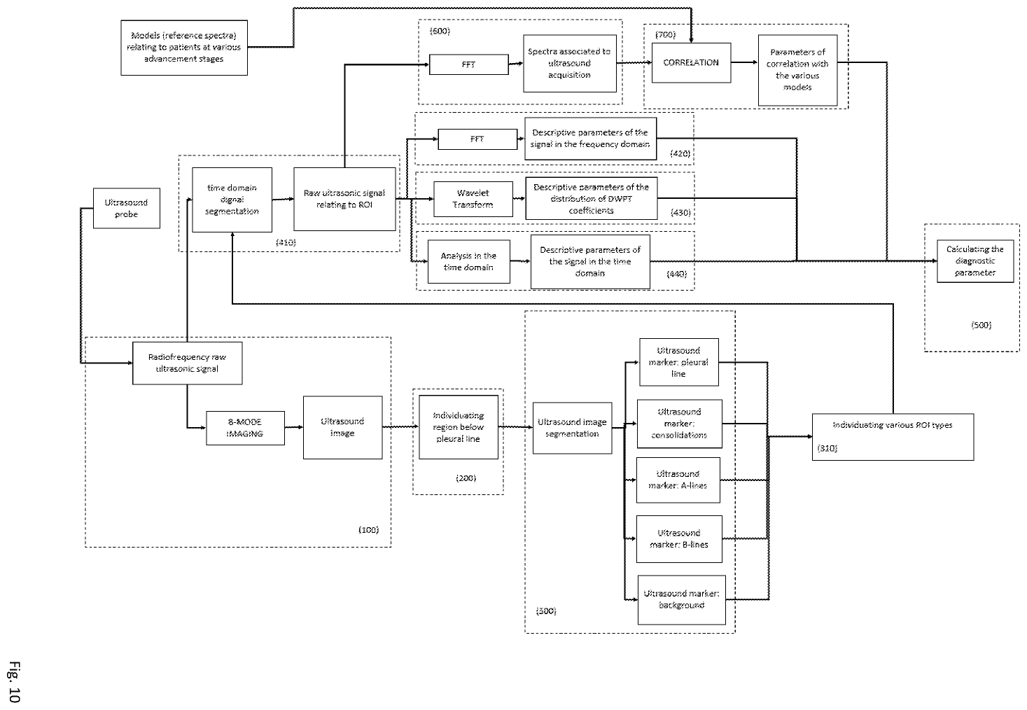

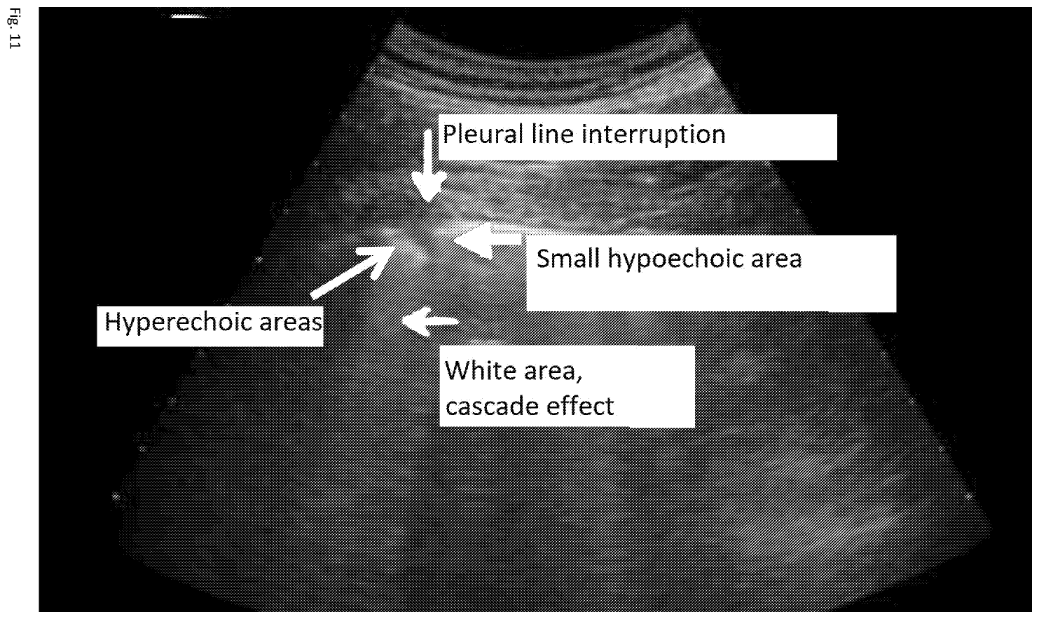

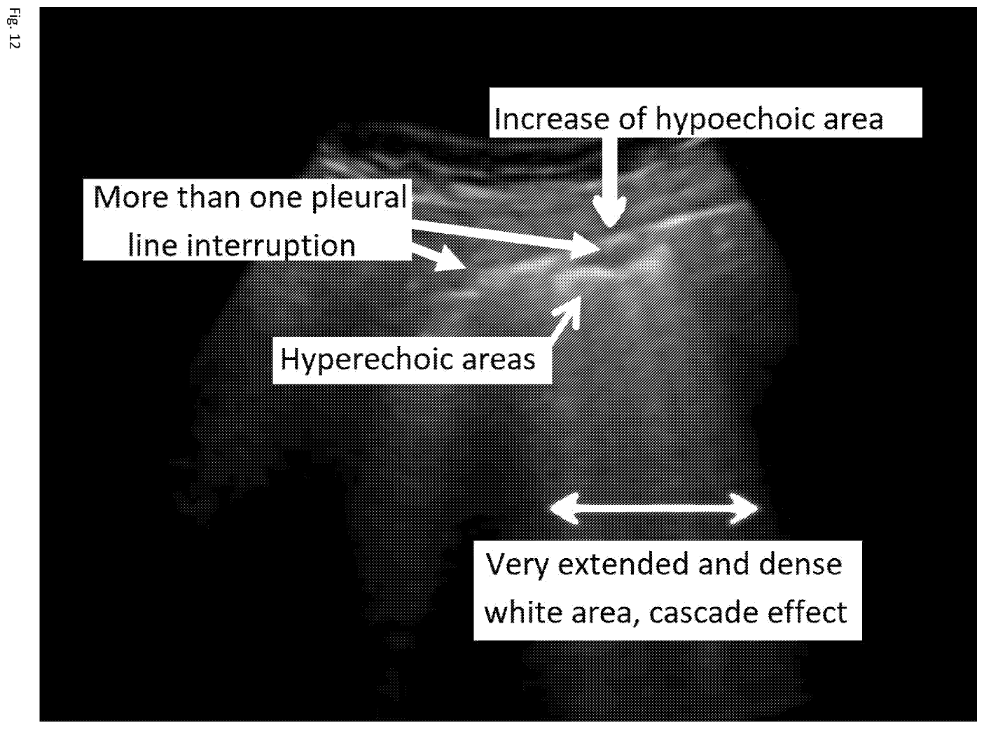

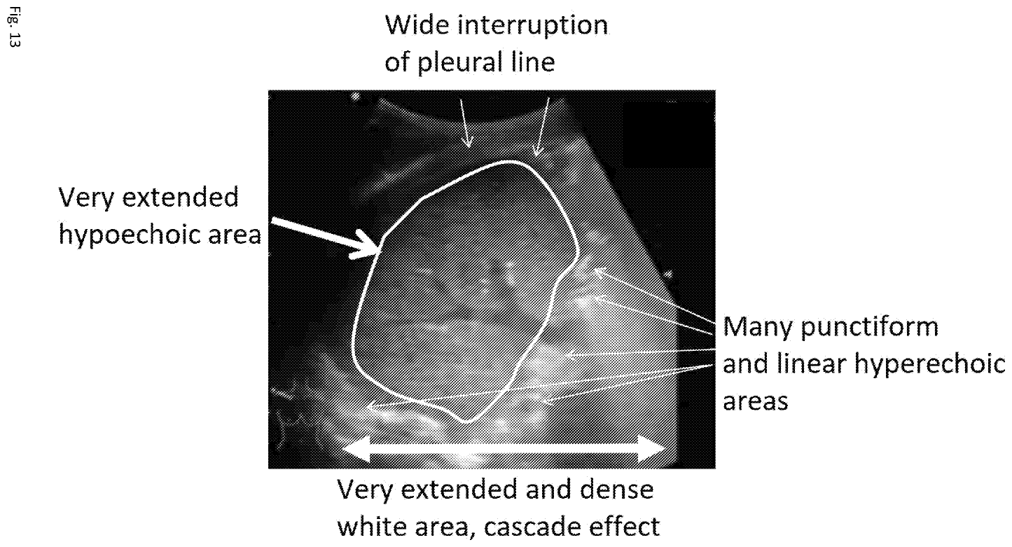

C4: consolidation areas (whose identification on a lung ultrasound image is shown in FIGS. 1 and 2). The consolidation areas can be identified by means of an automatic segmentation algorithm which, after the pleural line is identified, carries out the steps of:
(i) identifying the pleural line interruptions (by identifying possible interruptions of the bright profile relating to the pleural perimeter);
(ii) identifying the lung consolidations by searching hypoechoic areas in sub-pleural tissues adjacent to the interruptions detected at point
(i);

(iii) identifying air bronchograms by searching possible punctiform and/or linear hyperechoic areas inside the consolidations detected at point (ii) or on their lower edge.

### C5: Background

Once the areas relating to A-lines, B-lines and consolidations are rejected from the ultrasound image, the sub-pleural background is the remaining portion positioned under the pleural line.

At the end of step (300), each acquired image has been segmented in a plurality of areas containing respective ultrasound markers (pleural line, possible A-lines, possible B-lines, possible consolidations, background).

Then, the method comprises the step: (500) calculating a diagnostic parameter representing the progression stage of a pneumonia as a function of a plurality of parameters characteristic of the correlation of the frequency spectra relating to regions of interest individuated on the ultrasound image with frequency spectra relating to regions of interest of the same type and relating to patients, whose disease advancement stage is known. In the following, the individuation of Regions of interest as a function of the ultrasound image segmentation is described.

According to a first embodiment, said diagnostic parameter is expressed by means of the classification of the pneumonia in an advancement class chosen among five (or more) increasing severity classes, the first one of which corresponding to the absence of the disease.

Preferably, but not limitingly, the method comprises further the step of:
(800) repeating the calculation of the diagnostic parameter of step (500) for a plurality of acquired ultrasound images, for the same patient, in a plurality of positions, thus obtaining a plurality of diagnostic parameters, each one associated to a corresponding acquiring position;
(900) defining another diagnostic parameter indicating the aeration of lungs and the disease severity as a function of said plurality of diagnostic parameters calculated at point (800). Preferably, said plurality of acquiring positions of point (800) comprises one or more of the following ones, and preferably all the following positions:
1. right lung back portion scan, lower quadrant;
2. right lung back portion scan, middle quadrant;
3. right lung back portion scan, higher quadrant;
4. left lung back portion scan, lower quadrant;
5. left lung back portion scan, middle quadrant;
6. left lung back portion scan, higher quadrant;
7. right lung sub-axillary/lateral portion scan, lower quadrant;
8. right lung sub-axillary/lateral portion scan, higher quadrant;
9. left lung sub-axillary/lateral portion scan, lower quadrant;
10. left lung sub-axillary/lateral portion scan, higher quadrant;
11. right lung front portion scan, lower quadrant;
12. right lung front portion scan, higher quadrant;
13. left lung front portion scan, lower quadrant;
14. left lung front portion scan, higher quadrant. Moreover, said further diagnostic parameter indicating the lung aeration and the disease severity of point (900) is calculated as percentage of the acquiring positions for which the relative diagnostic parameter is of “healthy” type (i.e. classified in the class of absence of disease).

In another embodiment, said further diagnostic parameter indicating the lung aeration and the disease severity of point (900) is calculated as the average of the diagnostic parameter relating to each acquiring position, calculated according to one of the methods described in the following.

In another embodiment, said further diagnostic parameter indicating the lung aeration and the disease severity of point (900) is calculated as weighted average of the diagnostic parameter relating to each acquiring position, calculated according to one of the methods described in the document and in which to the diagnostic parameter calculated for each one of said acquiring positions a weight proportional is given to the lung volume which can be acquired from the respective acquiring position. Preferably, after step (900), the following steps are carried out:
(910) defining an asymmetry parameter of the disease severity;
(920) defining the pneumonia as not caused by Sars-Cov-2 virus, if said asymmetry parameter is higher than a predetermined threshold.

In a first embodiment, said asymmetry parameter is calculated as the difference between the sum of Pneumonia Scores calculated for the acquisition positions relating to a lung and the sum of Pneumonia Scores calculated for the acquisition positions relating to the other lung. In another embodiment, said asymmetry parameter is calculated as the ratio between the sum of Pneumonia Scores calculated for each acquisition position relating to a lung and the sum of Pneumonia Scores calculated for each acquisition positions relating to the other lung.

With regard to the ROI segmentation in the time domain, it is to be specified that, while in case of bone tissues, for which at the state of the art a method of diagnosis based on the ultrasound signal frequency analysis was described, the segmentation in time is a process independent of any hypothesis about the healthy condition of a patient and the identification of the interface of the target bone structure with respect to soft tissues always occurs in the same manner, in case of the method according to the present invention applied to lung ultrasound, to carry out the ROI segmentation in the time domain, it is needed:
to segment the markers defined at point (300) on the image (pleural line, and in case the pleural line is not continuous, its portions; where present, the markers relating to A-lines, B-lines, consolidations; background); to define at least one Region of Interest (ROI) as a function of such segmentation; for each raw ultrasound signal associated to each ultrasound image segment contained inside said at least one Region on Interest, to calculate the frequency spectrum and to associate to the spectrum the information relative to the ROI type the spectrum refers to.

After step (300), the method comprises then the step of:

(310) individuating at least one region of interest as a function of the type, quantity and configuration of the ultrasound markers individuated at point (300).

In order to individuate said at least one region of interest of point (310), the following cases are distinguished as a function of the ultrasound image segmentation results, carried out at point (300). Case 1) continuous pleural line and visible A-lines: the image portion between pleura and first A-line is considered as ROI (type 1 ROI);

Case 2) continuous pleural line and not visible A-lines: the image portion between pleura and the depth at which in the time domain the signal has an amplitude with respect to the peak amplitude produced by the pleura reflection at least equal to 5% or 10% is considered as ROI (type 2 ROI); Case 3) discontinuous pleural line without visible artifacts (neither A-lines nor B-lines): a plurality of ROIs is considered, each one corresponding to a tract where pleura is continuous. Each one of these ROIs is defined as in case 2 (type 1 or 2 ROI);

Case 4) discontinuous pleural line and visible B-lines: a plurality of ROIs is considered. In particular:
(i) possible continuous pleural line tracts are treated as in case 1) or case 2), depending on whether A-lines are visible or not (type 1 or 2 ROI);
(ii) each area identified by an isolated B-line or by more coalescent B-lines is considered as another ROI (type 3 ROI);

Case 5) presence of consolidations: in addition to the ROIs individuated in case 1 to 4, a plurality of ROIs, each one corresponding to a relative consolidation, is considered as resulting from the image segmentation process of point (300) (type 4 ROI).

To each region of interest, a variable describing the type of the identified region of interest is associated as well, among the just described ones. Therefore, the method according to the invention, after step (310) and before step (500), comprises (cfr. scheme of FIG. 7) the following steps:

(410) extracting from each raw ultrasonic signal received by each one of said CMUT or piezoelectric transducers of step (100) the portion corresponding to the relative segment of ultrasound image contained in each ROI individuated at point (300).

It is to be repeated that the segmentation of the raw ultrasonic signal is carried out in the time domain and the segmented signal is the raw one, i.e. received by the ultrasonic probe and not yet object of the processing the ultrasound image is obtained with.

This step allows to obtain a first important result: all and only the information relating to the signal generating the image of each ROI are contained in the thus segmented signal, since the raw signal has not been processed yet, with the result of losing information.

At the end of the segmentation process, it will be then obtained, for each acquisition and for each ROI, a matrix of P×N dimensions, where P is the number of points of RF raw signal corresponding to each ROI individuated at point (300) and N is the number of ultrasonic signals present in the ROI width. In the extreme case N is equal, at most, to the number of view lines generated by the piezoelectric transducers provided in the ultrasound probe used in the considered image. The P value will be instead a function of the individuated ROI depth.

Preferably, but not limitingly, after step (410) and before (420), the method comprises the step of:

(415) filtering each signal extracted at point (410) with a band-pass filter.

Preferably, the passing band is between 1 and 18 MHZ, but different extensions of the frequency band can be used to adapt better the procedure to different probe characteristics. Downstream of the filtering then, for each acquisition, it will be obtained a matrix of P×N dimensions, the same dimensions as the matrix obtained at the end of the segmentation process. Hence, the method comprises the step of:

(420) carrying out an analysis in the frequency domain of each raw ultrasonic signal extracted at point (410) by extracting a set of parameters characteristic of the signal in the frequency domain.

Said set of parameters characteristic of the signal in the frequency domain defined at point (420) is calculated after calculating for each raw signal extracted at point (410) the FFT (Fast Fourier Transform) of the signal, thus obtaining N frequency spectra associated to each ROI individuated at point (310).

Preferably, but not limitingly, said second set of parameters characteristic of the signal in the frequency domain is calculated after:

(i) calculating the module in dB of the absolute value of each one of said N frequency spectra, according to the formula:

Spectrum dB=20*log10(abs (spectrum)) thus obtaining still N spectra;
(ii) calculating the average spectrum (by average of all the values relating to the same frequency) of the spectra obtained at point (i), thus obtaining an average spectrum of ROI.

Preferably, said spectrum is calculated in a frequency range between 1 and 5 MHz; in another embodiment, said spectrum is calculated in a frequency range between 6 and 12 MHz; in another embodiment, said spectrum is calculated in a frequency range between 10 and 18 MHz.

On the thus obtained average spectrum of each ROI, a set of parameters characteristic of signal in the frequency domain defined at point (420) is calculated, which comprises one or more of the following parameters: a) the maximum value (PEAK) of said average spectrum (dimensions 1×1); b) the area of the spectrum, obtained by calculating the integral of the spectrum on the axis of frequencies in a determined frequency range (dimensions 1×1); c) spectrum peak frequency, i.e. frequency for which the spectrum has its own maximum (dimensions I×I), d) −6 dB band start frequency (lowest frequency of the spectrum having value equal to −6 dB, after having normalized the average spectrum with peak at OdB) (dimensions 1×1); e) −6 dB band end frequency at −6 dB (highest frequency of the spectrum having value equal to −6 dB, after having normalized the average spectrum with peak at OdB) (dimensions 1×1); f) band width at −6 dB (difference expressed in Hz between −6 db band end frequency and −6 dB band start frequency) (dimensions 1×1); g) spectrum slope (derived with respect to the frequency) calculated at a determined frequency; h) coefficients of a polynomial interpolating said average spectrum in a frequency range comprising said peak frequency.

At step (500), as yet said, a diagnostic parameter is calculated, which represents the progression staging of a pneumonia as a function of the set of parameters characteristic of the signal in the frequency domain defined at point (420) and relating to each one of said ROIs individuated at point (310).

By means of the description of the method for calculating the diagnostic parameter of point (500), it is to be specified what follows.

In a first embodiment, said diagnostic parameter is a numeric value representing the pneumonia severity. Conveniently, said numeric value can be expressed in a scale from 0 to 100, and called for simplicity Pneumonia Score in the following. In a first embodiment, the Pneumonia Score is calculated by using a regression function associating to a set of numeric values characteristic of the correlation of the spectra associated to the regions of interest of each type individuated at step (310) with spectra relating to regions of interest of the same type and relating to patients whose disease advancement stage is known, a Pneumonia score numeric value:

Pneumonia score=f(Coriₐ, . . . , Coriₑ, . . . , Cor4a, Cor4e,) Where the subscripts Cor±j indicate the type of region on interest individuated and the disease advancement class, respectively.

Conveniently, said regression function f is estimated by using a set of parameters calculated in the same manner, relating to lung ultrasound images of a plurality of patients whose disease advancement stage is known and for whom the class of belonging has been defined by “skilled operators”, as a function of the analysis of the ultrasound scans, and/or as a function of the information derived from other diagnostic examinations, as for example CT.

In a second embodiment, the Pneumonia Score is calculated by using a regression neural network to which the correlation parameter values (Coriₐ, . . . , Corie, . . . , Cor4a, Cor4e) are provided in input, and which provides in output the diagnostic parameter value. The neural network is trained by using a set of parameters relating to lung ultrasound images of a plurality of patients whose disease advancement stage is known and for whom the class of belonging has been defined by “skilled operators”, as a function of the ultrasound scans analysis and/or as a function of information derived from other diagnostic examinations, as for example CT.

In an embodiment, a neural classification network can be used, to which the correlation parameter values (Coriₐ, . . . , Coriₑ, . . . , Cor4a, Cor4e) are provided in input, and configured to provide in output a vector containing the probability of belonging to each class of disease advancement, wherein an interval of Pneumonia Score values is assigned to each class. Pneumonia Score is then calculated as a function of the probability of belonging to each class and of the values defining the lower and upper ends of each class. For example, Pneumonia Score can be calculated as a function of the ends of the first and second class for probability of belonging, weighted as a function of the respective probabilities of belonging.

If, for example, the classes are defined as in the following table;

| | Lower | Higher | |
|---|---|---|---|
| | Pneumonia | Pneumonia | |
| Class | Score | Score | Description |
| 1 | 0 | 20 | Disease absent |
| 2 | 20 | 40 | Disease in initial |
| | | | stage |
| 3 | 40 | 60 | Disease in |
| | | | intermediate stage |
| 4 | 60 | 80 | Disease in advanced |
| | | | stage |
| 5 | 80 | 100 | Disease in peak stage |

The following condition can occur, in which the classification neural network provides in output the vector indicated in the “Probability of belonging” column.

| | Lower | Higher | | |
|---|---|---|---|---|
| | Pneumonia | pneumonia | | Probability |
| Class | Score | Score | Description | of belonging |
| 1 | 0 | 20 | Disease absent | 0.13 |
| 2 | 20 | 40 | Disease in | 0.37 |
| | | | initial stage | |
| 3 | 40 | 60 | Disease in | 0.25 |
| | | | intermediate | |
| | | | stage | |
| 4 | 60 | 80 | Disease in | 0.17 |
| | | | advanced stage | |
| 5 | 80 | 100 | Disease in | 0.08 |
| | | | peak stage | |

The first two classes, in order of probability of belonging are class 2 (initial; Pneumonia Score between 20 and 40; probability 0.37) and class 3 (intermediate; Pneumonia Score between 40 and 60; probability 0.25). So, the Pneumonia Score can be calculated as the weighted average of the outer intervals of the two classes of variability, weighted with the respective probabilities of belonging.

$$Pneumonia Score = ( 0.25 \times 60 + 0.37 \times 20 ) / ( 0.25 + 0.37 ) = 36.1$$

As yet said, the regression or classification neural network has to be suitably trained, according to techniques known per se at the state of the art, by using a training set of features relating to images, which:
(i) are acquired and processed by means of the same just described modes;
(ii) are relating to patients, whose exact classification in terms of disease staging is previously known.

The exact classification in terms of disease staging can be known in various manners. As a way of pure example, many patients and during the same day can be subjected to the same examination by means of other diagnostic techniques (radiography, high resolution CT) and on the basis of these examinations the exact disease staging can be defined, in this case using automatic or semi automatic dedicated software; alternatively, on the lung ultrasound images obtained for the same patients and during the same day, one or more skilled ultrasound operator can carry out a manual classification of the disease (i.e. based on the analysis of images in light of one's expertise and on the possible extraction of quantitative parameters carried out manually on the images).

The effective network training can be evaluated, according to techniques known at the state of the art, by means of data relating to a “validation set” (relating to patients whose exact classification is known, but whose data are not used for the network training). Yet, the structure of the neural network could be designed and optimized as well according to techniques known at the state of the art, and different configurations of neural networks can be used without departing from the scope of the invention.

In the following, the raw ultrasonic signal analysis operations are described in detail, which are needed to allow the calculations of a diagnostic parameter representing the progression stage of a pneumonia, by using a plurality of parameters extracted from the radiofrequency raw ultrasonic signal, analyzed in the time domain, and/or frequency domain, and/or by using Wavelet transforms.

It is to be specified that in all the embodiments described in the following (cfr. step 410), the radiofrequency raw ultrasonic signal is segmented in the time domain in order to extract only the portions relating to the regions of interest extracted as a function of the ultrasound markers type individuated at step (300). The thus extracted signal contains all the information relating to the portions under the pleural line individuated as regions of interest, also those ones normally lost in the following processing needed to obtain the ultrasound image: this is a further characteristic of the method according to the present invention distinguishing it from all the diagnostic methods known at the state of the art, based instead on the analysis of ultrasound images.

Moreover, in the specific case of lung ultrasound, it is not known a priori how many regions of interest will be detected and their type, since it is not known a priori how many and which ultrasound markers will be individuated in the ultrasound image. According to another embodiment (cfr. scheme of FIG. 8), after step (310) and before step (500), the method comprises further the yet defined step (410), in which the signal is segmented in the time domain, and the step of:

(430) carrying out a Wavelet analysis of the signal extracted at point (410) and obtaining a set of parameters comprising one or more statistical parameters relating to the distribution of DWPT (Discrete Wavelet Packet Transform) coefficients, relating to the signal segments relating to each individuated ROI.

In this embodiment, the method is characterized in that at step (500) a diagnostic parameter is calculated, representing the progression stage of a pneumonia as a function of the set of parameters comprising one or more statistical parameters relating to the distribution of DWPT coefficients (Discrete Wavelet Packet Transform) defined at point (430). Preferably, for each signal extracted at point (410) the Wavelet analysis is carried out up to the third level, which from each signal segment considered generates 8 DWPT coefficients, a statistical distribution of the values assumed in ROI being associated thereto, which can be characterized by average, standard deviation, skewness, kurtosis values.

According to another embodiment (cfr. scheme of FIG. 9), after step (410) the method comprises further the step of:

(440) carrying out an analysis in the time domain of each raw ultrasonic signal extracted at point (410) by extracting a set of parameters characteristic of the signal in the time domain.

In this embodiment, the method is characterized in that at step (500) a diagnostic parameter representing the progression stage of a pneumonia is calculated as a function of the set of parameters comprising one or more parameters characteristic of the signal in the time domain defined at point (440).

Preferably, said set of parameters characteristic of the signal in the time domain defined at point (440) comprises one or more of the following parameters: a) the average value of the raw ultrasonic signal, obtained as the average of the absolute value of the raw ultrasonic signal for all said signals extracted at step (410); b) one or more of the following values, characteristic of the matrix obtained calculating the absolute value of the radiofrequency ultrasonic signal relating to each transducer. The matrix obtained is of P×N dimensions, according to what above explained. Said characteristic values, whose formulation is known at the state of the art for other purposes, comprise: entropy of the matrix, homogeneity of the matrix; energy of the matrix; contrast of the matrix. In this description, the mathematical formulation for calculating said parameters in a P×N matrix is not reported, since it is the same as the one known at the state of the art. In order to allow the comparison of the frequency spectra, after step (410) and before step (500), the method according to the invention comprises the following steps:
(450) extracting frequency spectra relating to the raw ultrasonic signal corresponding to a segment of the ultrasound image contained in each ROI individuated at step (310), associating to each spectrum the information relating to the ROI type.
(470) comparing each one of said spectra extracted at point (450) with relative reference spectra (models), relating to ROIs of the same type and calculated for a healthy patient and for patients suffering from pneumonia at various advancement stages, in order to calculate a plurality of parameters characteristic of the correlation of said spectra extracted at point (450) with said reference spectra.

The correlation is calculated between spectra referred to Regions of Interest of the same type.

It is clear that not for all the advancement classes of the disease, it is possible to calculate all the types of reference spectrum. For example, spectra relating to consolidations will not be present for healthy patients.

If a determined type of region of interest was not individuated, the relative coefficient of correlation would be excluded from the calculation. For example, in case of absent reference spectrum, the coefficient of correlation could be equal to 1 if absent also among the reference spectra relating to a specific advancement class of the disease, and equal to 0 if present among the spectra relating to a specific advancement class of disease.

The method is characterized in that at step (500) it is calculated a diagnostic parameter representing the progression stage of a pneumonia as a function of said plurality of parameters characteristic of the correlation calculated at point (470).

It is to be specified that in the present document, the word “model” and the definition “reference spectrum” are used with the same meaning. It is to be said firstly, that preferably said plurality of reference spectra (or models) comprises, for each type of Region of Interest:
a model relating to a healthy patient; a model relating to a patient with initial stage disease; —a model relating to a patient with intermediate stage disease;
a model relating to a patient with advanced stage disease;
a model relating to a patient with peak stage disease.

Conveniently, the ultrasound system according to the invention is configured to store said plurality of reference spectra. In a preferred embodiment, the method provides further to calculate each one of said reference spectra (models), according to the following steps.

For a patient with disease in any advancement class:
(i) acquiring at least an ultrasound image, and preferably a plurality of ultrasound images, of the lung of the patient, in which at least the pleural line and lung portion below it are visible;
(ii) individuating on said ultrasound images a plurality of regions of interest according to what described in the step (310);
(iii) segmenting, in the time domain, the radiofrequency raw ultrasonic signal in order to extract the signal portions corresponding to the ultrasound image segments contained in each ROI; (iv) calculating, for each one of said signal portions extracted at point (iii) the frequency transform (FFT) in order to obtain a frequency spectrum corresponding to each extracted signal;
(v) normalizing each spectrum with respect to its maximum value, so that its maximum value is 0 dB;
(vi) calculating the average of all the normalized spectra of point (v) and relating to the same type of ROI, thus obtaining an average reference spectrum relating to a healthy patient (or relating to a patient with disease in anyone of the three reference classes) for each type of Region of Interest.

Preferably, the just described steps can be carried out for a plurality of healthy patients (or with disease in anyone of the three reference classes), considering the normalized spectra relating to each patient, for the calculation. Moreover, downstream of point (vi) the method provides the steps of:
(vii) calculating the coefficient of correlation between each normalized spectrum of point (v) and the relating average reference spectrum of point (vi);
(viii) selecting, among all the normalized spectra of point (v), those ones having a coefficient of correlation (r) with said average spectrum of point (vi) greater than 0.900, thus rejecting the other spectra;
(ix) calculating a new average of the normalized spectra extracted at point (viii), thus obtaining a new average reference spectrum relating to a healthy patient (or relating to a patient with disease in anyone of the other reference classes) for each type of ROI;
(x) calculating the coefficient of correlation between each normalized spectrum extracted at point (viii) and said new average reference spectrum of point (ix), and selecting the spectra having a coefficient of correlation (r) with said new average spectrum greater than 0.900, thus rejecting the other spectra;
(xi) repeating, iteratively, points (ix) and (x) up to when all the spectra remained have a coefficient of correlation (r) with the average spectrum greater than 0.900;
(xii) calculating the average of the not rejected spectra, thus obtaining a final average reference spectrum relating to a healthy patient (or relating to a patient with disease in anyone of the other reference classes) for each ROI type.

With reference to point (450) relating to the spectrum extraction, it is to be specified that, preferably, said spectrum is filtered with a band pass filter. Preferably, the passing band is between 1 and 18 MHz, but different extensions of the frequency band can be used to adapt better the procedure to different ultrasound probe characteristics.

It is to be specified that in a first preferred embodiment, the spectrum is extracted, relating to the segment contained in the ROI of each one of the signals received by each piezoelectric transducer included in the piezoelectric transducer array of the ultrasound probe. In other words, each radiofrequency ultrasonic signal is segmented in the time domain to extract its portion relating to the Region of Interest (which in a preferred embodiment is the whole portion of image positioned below the pleural line), and the frequency spectrum is calculated from the thus extracted portion.

Preferably, after step (450) and before step (470), the method provides further the step of:

(460) calculating the average of all the spectra extracted at point (450) and relating to each type of ROI, in order to obtain an average spectrum representing each ROI type.

The value obtained as average of the values for the same frequency of all the spectra extracted at point (450) is associated to each frequency of the average spectrum. Said average spectrum can be calculated also as average of the spectra extracted according to the just described modes and relating to a plurality of following ultrasound acquisitions, i.e. a sequence of ultrasound frames acquired as a sequence with the probe being still and so relating to the same anatomical region. Preferably, moreover, the method provides the step of carrying out a step of frequency spectrum compensation, carried out on said plurality of spectra extracted at point (450) or on said average spectrum calculated at point (460), multiplying the value relating to each frequency by a value depending on the transfer function of the used ultrasound probe.

With reference to point (470), relating to the comparison between the spectrum relating to the patient and the model, in a first embodiment, the comparison occurs by calculating the coefficient of correlation, on the whole frequency range, between each spectrum extracted at point (450) and said models defined at point (470) for ROIs of the same type; alternatively, the comparison occurs between said average spectrum calculated at point (460) and said models defined at point (470). Preferably, the comparison occurs by means of the calculation of the coefficient of correlation in a frequency range between 1 and 5 MHz in case of convex type probe, and in a frequency range between 6 and 18 MHz for a linear type probe.

In a first embodiment, said plurality of parameters characteristic of the correlation of said at least one spectrum with said reference spectra comprises the coefficient of correlation of the average spectrum referred to each region of interest with each reference spectrum relating to ROIs of the same type for patients at various disease advancement stage.

In another embodiment, for each spectrum extracted at point (450) the coefficient of correlation with each reference spectrum referred to ROIs of the same type is calculated, and each spectrum is then defined as healthy, initial, intermediate, advanced or peak spectrum depending on which one is the maximum coefficient of correlation between the various calculated coefficients of correlation (a spectrum for which the coefficient of correlation with the healthy reference spectrum is maximum will be defined as “healthy spectrum”, etc.).

In this case, said plurality of parameters characteristic of the correlation of said at least one spectrum with said reference spectra comprises the percentage value of the spectra of each type (healthy spectra, initial spectra, intermediate spectra, advanced spectra, peak spectra) with respect to the whole spectra extracted at point (450).

In this case, the diagnostic parameter can be calculated as a function of the ends of the first and second class for which the greater percentage of the spectra are present, weighted as a function of the respective coefficients of correlation.

The following situation can occur.

| | Lower | Higher | | |
|---|---|---|---|---|
| | Pneumonia | Pneumonia | | Classified |
| Class | Score | Score | Description | spectra % |
| 1 | 0 | 20 | Disease absent | 5 |
| 2 | 20 | 40 | Disease in | 10 |
| | | | initial stage | |
| 3 | 40 | 60 | Disease in | 65 |
| | | | intermediate | |
| | | | stage | |
| 4 | 60 | 80 | Disease in | 15 |
| | | | advanced stage | |
| 5 | 80 | 100 | Disease in peak | 5 |
| | | | stage | |

The first two classes, in order of decreasing coefficient of correlation, are class 3 (intermediate; Pneumonia Score between 40 and 60; 65%) and class 4 (advanced; Pneumonia Score between 60 and 80; 15%). The Pneumonia Score can be then calculated as the weighted average of the outer intervals of the two classes, weighted with the respective % of classified spectra.

$$Pneumonia score = ( 0 , 65 \times 40 + 0.15 * 80 ) / ( 0.65 + 0.15 ) = 47.5$$

In another embodiment, the used ultrasound probe comprises a double array of piezoelectric or cMUT transducers, each one configured to work at a respective nominal frequency, the two nominal frequencies being distinguished between each other so that the frequency spectra of the ultrasonic signals emitted by the two transducers are not overlapping. In a first embodiment, said nominal frequencies are 3 MHz and 10 MHz, respectively.

In this case, the device is configured to acquire a first ultrasound image by using the array of piezoelectric transducers having the highest nominal frequency.

On said ultrasound image:
the pleural line continuity and reflectivity is verified,
a segmentation is carried out in order to detect the ultrasound markers in the portion of image for which the intensity of the reflected signal is greater at a predetermined percentage (for example 90%) of the intensity of the signal reflected from the layer made up of the skin up to the pleura included.

Therefore, a second ultrasound image is acquired by using the piezoelectric transducers array having the lower nominal frequency, on which the markers relating to greater depths are segmented.

Preferably, moreover, the method comprises the acquisition of at least one image (and preferably a plurality of images) with the probe parallel to the ribs, so that an intercostal acquisition is carried out, and then the acquisition is repeated with the probe positioned in the same acquisition point, but rotated of 90°, in orthogonal direction to the ribs. Both the images are then analyzed according to what described and the diagnostic parameter is calculated as a function of the correlation of the spectra acquired during the two acquisitions.

1. A method for calculating a diagnostic parameter indicating the stage of a pneumonia, comprising the steps of:
(100) acquiring, by means of an ultrasound device provided with a probe comprising an array of CMUT or piezoelectric transducers each configured to emit an ultrasonic impulse directed to the tissues object of classification and to receive the raw ultrasonic signal reflected by the tissues in response to said ultrasonic impulse, at least an ultrasound image of the lung of a patient, in which it is visible at least the pleural line and a portion of lung below it;
(200) individuating, inside said at least one image acquired at point (100), the region under the pleural line;
(300) segmenting said region under the pleural line in order to individuate a set of ultrasound makers (Ci, . . . , Cn) therein, relating to the pleural line (Ci), A-lines (C2), B-lines (C3), consolidations
(c4); characterized in that it comprises further the steps of: (310) individuating at least a region of interest (ROI) as a function of the type, quantity and configuration of the ultrasound markers individuated at step (300) and associating said at least one region of interest to a specific ROI type;
(450) extracting frequency spectra relating to the raw ultrasonic signal corresponding to segments of the ultrasound image contained in each ROI individuated at step (310), associating to each spectrum the information relating to the individuated ROI type;
(470) comparing each one of said spectra extracted at point (450) with relative reference spectra, relating to ROIs of the same type and calculated for healthy patients and for patients suffering from pneumonia at various advancement stages, in order to calculate a plurality of parameters characteristic of the correlation of said spectra extracted at point (450) with said reference spectra;
(500) calculating a diagnostic parameter representing the progression stage of a pneumonia as a function of said plurality of parameters characteristic of the correlation calculated at point (470), said diagnostic parameter being calculated by means of a regressor associating to said plurality of parameters of correlation a value of the diagnostic parameter.

2. The method for calculating a diagnostic parameter according to claim 1, wherein said regressor is a function of regression associating to a set of numeric values characteristic of the correlation of the spectra associated to the regions of interest of each one individuated at step (310) with spectra relating to regions of interest of the same type and relating to patients whose disease advancement stage is known, a numeric value of the diagnostic parameter:
$$Pneumonia score = f ( Coria , … , Corie , … , Cor 4 a , Cor 4 e , )$$

3. The method for calculating a diagnostic parameter according to claim 1, wherein said diagnostic parameter is calculated by using a regression neural network to which the correlation parameter values (Coria, . . . , Corie, . . . , Cor4a, Cor4e) are provided in input, and which provides in output the diagnostic parameter value.

4. The method for calculating a diagnostic parameter according to claim 1, wherein at step (310), in case at step (300) the pleural line is continuous and one or more A-lines are visible, the portion of image between pleura and first A-line is considered as ROI.

5. The method for calculating a diagnostic parameter according to claim 1, wherein at step (310), in case at step (300) the pleural line is continuous and A-lines are not visible, the portion of image between pleura and the depth at which in the time domain the signal has an amplitude with respect to the peak amplitude produced by the pleura reflection at least equal to 5% is considered as ROI.

6. The method for calculating a diagnostic parameter according to claim 1, wherein at step (310), in case at step (300) the pleural line is individuated, the same being discontinuous and neither A-lines nor B-lines being visible, a plurality of ROIs is considered, each one corresponding to a tract where pleura is continuous, and for each one of them the portion of image between pleura and the depth at which in the time domain the signal has an amplitude with respect to the peak amplitude produced by the pleura reflection at least equal to 5% is considered as ROI.

7. The method for calculating a diagnostic parameter according to claim 1, wherein at step (310), in case at step (300) the pleural line is individuated, the same being discontinuous and at least a B-line being visible, a plurality of ROIs is considered:
(i) possible continuous pleural line tracts are treated as in the previous claims depending on whether “A-lines” are visible or not;
(ii) each area identified by an isolated B-line or by more coalescent B-lines is considered as another ROI.

8. The method for calculating a diagnostic parameter according to claim 1, wherein at step (310), in case at step (300) at least a consolidation is individuated, in addition to the yet individuated
ROIs further ROIs are considered, coincident with the area relating to each consolidation.

9. The method for calculating a diagnostic parameter according to claim 1, wherein said plurality of reference spectra (or models) comprises, for each type of Region of Interest:
a model relating to a healthy patient;
a model relating to a patient with initial stage disease;
a model relating to a patient with intermediate stage disease;
a model relating to a patient with advanced stage disease;
a model relating to a patient with peak stage disease.

10. The method for calculating a diagnostic parameter according to claim 1, wherein at step (450), a plurality of frequency spectra are extracted, relating to the raw ultrasonic signal corresponding to a plurality of respective segments of the ultrasound image contained in each ROI, in that, after step (450) and before step (470), it comprises the step of:
(460) calculating the average of all the spectra extracted at point (450) and relating to each type of ROI, in order to obtain an average spectrum representing each ROI type, and in that at point (470) each average spectrum representing each ROI is compared with a reference spectrum relating to a healthy patient for a ROI of the same type and with a plurality of reference spectra relating to patients suffering from pneumonia at various advancement stages, as well for the same
ROI type, in order to calculate a plurality of parameters characteristic of the correlation of said average spectrum with said reference spectra relating to ROIs of the same type, and in that the diagnostic parameter calculated at point (500) is calculated as a function of said plurality of parameters characteristic of the correlation of said average spectrum with said reference spectra relating to ROIs of the same type.

11. The method for calculating a diagnostic parameter according to claim 10, wherein at point (470) the comparison occurs by calculating the coefficient of correlation, on the whole frequency range, between each spectrum extracted at point (450) and said reference spectra relating to patients suffering from pneumonia at various advancement stages.

12. The method for calculating a diagnostic parameter according to claim 10, wherein said plurality of parameters characteristic of the correlation of said at least one spectrum with said reference spectra comprises the coefficient of correlation of said average spectrum representing ROI calculated at point (460) with each one of said reference spectra, in that to each one of said classes corresponding to patients suffering from pneumonia at various advancement stages an interval of variability is associated of the diagnostic parameter between a lower end and an upper end, and in that said diagnostic parameter is calculated as a function of the ends of the first and second class for the value of coefficient of correlation, weighted as a function of the respective coefficients of correlation.

13. The method for calculating a diagnostic parameter according to claim 12, wherein for each spectrum extracted at point (450) the coefficient of correlation is calculated with each of said reference spectra, in that each spectrum extracted is then defined as healthy, initial, intermediate, advanced or peak spectrum depending on which one is the maximum coefficient of correlation between the various calculated coefficients of correlation, and in that said plurality of parameters characteristic of the correlation of said at least one spectrum with said reference spectra comprises the percentage value of the spectra of each type (healthy spectra, initial spectra, intermediate spectra, advanced spectra, peak spectra) with respect to the whole spectra extracted at point (450).

14. An ultrasound device comprising computing means on which computer programs are loaded, configured to carry out the method according to claim 1.
